(19) United States
(12) Patent Application Publication
(10) Pub. No.: US 2010/0143271 A1
(43) Pub. Date: Jun. 10, 2010

(54) Method for preparing amorphous calcium phosphate and oral care composition containing amorphous calcium phosphate prepared by the same

(75) Inventors: Jen-Chang Yang, Taipei City (TW); Dian-Yu Ji, Taipei City (TW); Chien-Chung Chen, Taipei City (TW); Haw-Ming Huang, Taipei City (TW); Sheng-Yang Lee, Taipei City (TW)

Correspondence Address: BACON & THOMAS, PLLC, 625 SLATERS LANE, FOURTH FLOOR, ALEXANDRIA, VA, 22314-1176, US

(73) Assignee: Taipei Medical University, Taipei (TW)

(21) Appl. No.: 12/461,942

(22) Filed: Aug. 28, 2009

(30) Foreign Application Priority Data
Dec. 10, 2008 (TW) 097147979

(51) Int. Cl. A61K 8/21; A61Q 11/00; C01B 25/26

(52) U.S. Cl. 424/52; 423/307

(57) ABSTRACT

A method for preparing amorphous calcium phosphate (ACP) and oral care composition containing ACP prepared by the same are disclosed, wherein the method comprises the following steps: (a) cleaning and pulverizing the oyster shells; (b) sintering the oyster shells to obtain a calcium-containing powder; (c) dissolving the calcium-containing powder in water to obtain a calcium-containing solution; and (d) mixing the calcium-containing solution with a other phosphate-containing solution, to produce amorphous calcium phosphate.

The method of the present invention not only can simplify the process and reduce the cost, but also can solve the problem of environment pollution resulting from wasted oyster shells. In addition, the ACP prepared in accordance with the method of the present invention further comprises some trace elements, such as strontium (Sr) and fluoride (F), which can improve dental health.

### BACKGROUND OF THE INVENTION

1. Field of the Invention

The present invention relates to a method for preparing amorphous calcium phosphate (ACP) and an oral care composition containing amorphous calcium phosphate prepared by the method is described hereinafter. More particularly, the present invention relates to a method for preparing amorphous calcium phosphate by using oyster shells. Furthermore, an oral care composition containing amorphous calcium phosphate prepared by the method is also described hereinafter.

2. Description of Related Art

Oyster is one of the major marine products for many countries. After the harvesting of valuable oyster meat, the removed oyster shells were a leftover. Only a minor part of the oyster shells was reused as a settlement for rearing of oyster larvae, an artificial reef, a handicraft, a construction material, a reagent for fishpond soil improving, an animal vegetative feed, or a soil amelioration. Owing to the huge amount of unused oyster shells, most of them can only be handled like a waste and buried directly. However, the residual oyster meat and the organic material remaining in the waste oyster shells ferment and release bad odor after long term exposure to the sun or rain. Hence, the wasted oyster shells not only spoil the scenery of the surroundings, but also greatly deteriorate the environmental sanitation due to the leakage of rotten organic material containing wastewater into underground water. Therefore, if the wasted oyster shells can further be used for new value-added applications, the development of oyster industry can be improved and the pollution issue from wasted oyster shells can be solved.

One of the main components of the oyster shells is calcium carbonate, which is usually used as a calcium supplement for bone nutrient. The content of calcium carbonate in oyster shells is 95.994%. The remaining components are typical metal oxides, such as sodium oxide (Na₂O), magnesium oxide (MgO), and strontium oxide (SrO). The following table shows the content of each element in the oyster shells.

| Cation | Weight percentage (%) |
|---|---|
| Ca | 37.400 |
| Na | 0.594 |
| Mg | 0.269 |
| K | 0.012 |
| Fe | 0.034 |
| Al | 0.036 |
| Mn | 0.011 |
| Cu | 0.001 |
| Zn | 0.011 |
| Sr | 0.091 |
| Si | 0.023 |

The oyster shells containing abundant natural calcium source and various trace elements can be used in many fields for enhancing the economic value of the oyster shells but reducing the environment pollution.

For fully utilizing the calcium from the animal shells, many attempts for extracting calcium element as a mineral supplement in health food have been developed. JP 08-131121 disclosed a method to prepare the calcium powders from the various shells of a shellfish for health drink usage. First, the calcium oxide powders were obtained by sintering cleaned animal shell under 900-1400° C. Next, a calcium solution was prepared by dissolving the calcium powders into an acid solution. Finally, the high purity calcium carbonate powders can be precipitated out by introducing the CO₂ gas into this calcium solution until the concentration exceeding the solubility limit of CaCO₃ solution.

Furthermore, JP 05-236915 disclosed that a health drink with dissolved calcium powders from scallop shells is manufactured and currently provided to market. The calcium powder used in the aforementioned health drink is prepared by sterilizing clean scallop shells under 100° C. first. After the sterilized scallop shells are well dried, pulverized, and filtered, calcium powders for producing health drinks use can be obtained.

Following the similar processing concept for treating animal shells, many commercial products of calcium supplements can be derived from the sintered oyster shells. Besides, U.S. Pat. No. 5,409,714 and JP 10-087307 disclosed that sintered oyster shells can be used as a natural product with good germicidal activity due to their high alkalinity.

Unlike those direct applications of sintered oyster shells as an end product, this subject invention attempts to use them as a raw material to produce calcium based derivatives especially for oral care applications.

Due to low calcium-to-phosphate (Ca/P) ratio and high solubility, amorphous calcium phosphate is widely applied in oral care products. Typically amorphous calcium phosphate is produced by a titration method represented by the following chemical reaction:

3CaCl₂+2Na₂HPO₄+nH₂O→Ca₃(PO₄)₂.nH₂O+4NaCl+2HCl

Due to the formation of acidic by-product, hydrogen chloride (HCl), the low pH of reaction solution tends to enhance the crystallinity and reduce the solubility of calcium phosphate. To ensure the amorphous nature of synthesized calcium phosphate, a base, i.e. sodium hydroxide (NaOH), is usually added to neutralize the reaction solution acidity and control the pH within the range of 8.5˜12. However, the by-product salt obtained from neutralization will decrease the purity of amorphous calcium phosphate if it is not fully washed. In addition, the pH adjustment of the reaction solution needs to be carried on slowly to avoid drastically increasing solution temperature that resulted in increasing crystallinity of calcium phosphate. In a word, conventional method for preparing amorphous calcium phosphate exhibits the disadvantages of complicate manufacturing process, long reaction time, and possible salt residue.

Many attempts have been formulated amorphous calcium phosphate into oral care product applications. One study disclosed an oral liquid composition prepared by adding amorphous calcium phosphate into a drink or mouthwash for dental caries prevention. Another oral care related toothpaste comprises amorphous calcium phosphate and sodium fluoride (NaF).

It is known that trace elements like strontium (Sr) and fluoride (F) are effective in dental care. For instance, strontium ions can bind the hydroxyapatite on the teeth surface to form strontium-apatite complex. It can block or seal the openings of dentinal tubules, prevent fluid flow, and desensitize dentin. On the other hand, fluoride can complex with hydroxyapatite to form fluoroapatite, which can increase the acid and caries resistance of enamel. Fluoride adheres to teeth surface and slowly releases over time that promotes tooth remineralization. According to the reports of U.S. Public Health Service, the addition of 1 ppm fluoride into drinking water can reduce the incidence rate of caries about 50˜60%. Also, applying 2% of fluoride varnish over tooth surface can decrease caries incidence rate by about 40% in children.

It is desirable to prepare amorphous calcium phosphate from oyster shells, which can reduce the preparation time and the possible salt residue in the final products. Moreover, the trace element contenting in oyster shells can enhance the effectiveness on dental care. In addition, the extensive utilization of oyster shells in value-added applications is beneficial. The environmental pollution of wasted oyster shells can also be solved.

### SUMMARY OF THE INVENTION

The object of the present invention is to provide a method for preparing amorphous calcium phosphate in a simple, low-cost, and green production. The present invention also provides a method for preparing amorphous calcium phosphate and its derivatives containing trace elements of strontium and fluoride. Since the amorphous calcium phosphate prepared from the oyster shells is cost effective and contains many useful trace elements, so the amorphous calcium phosphate can be widely applied to the products for dental care to prevent carries and promote remineralization on teeth.

To achieve the objective, a method for preparing amorphous calcium phosphate of the present invention comprises the following steps: (a) cleaning and pulverizing the oyster shells; (b) sintering the oyster shells to obtain a calcium-containing powder; (c) dissolving the calcium-containing powder in water to obtain a calcium-containing solution; and (d) mixing the calcium-containing solution with a phosphate-containing solution, to produce amorphous calcium phosphate.

In addition, the present invention also provides an oral care composition, which comprises: amorphous calcium phosphate. Herein, the amorphous calcium phosphate is prepared in accordance with the following steps: (a) cleaning and pulverizing the oyster shells; (b) sintering the oyster shells to obtain a calcium-containing powder; (c) dissolving the calcium-containing powder in water to obtain a calcium-containing solution; and (d) mixing the calcium-containing solution with a phosphate-containing solution, to produce amorphous calcium phosphate containing trace element of strontium and fluoride.

The method of the present invention utilizes the abundant calcium source and trace elements in the oyster shells to prepare amorphous calcium phosphate for dental care purpose. In addition, the preparation of amorphous calcium phosphate in the present invention is robust and no need for pH adjustment. It's a simple, quick, and high yield process. The method of the present invention can avoid the formation of crystalline phase and salt residue. It can fully utilize the calcium, strontium, and fluoride component in the oyster shells to produce amorphous calcium phosphate for oral care applications, so it is possible to increase the economic value of the oyster shells and reduce environment pollution.

The method for preparing amorphous calcium phosphate and the oral care composition of the present invention may further comprise a step (c1) after the step (c): filtering the calcium-containing solution.

The method for preparing amorphous calcium phosphate and the oral care composition of the present invention may further comprise a step (d1) after the step (d): filtering the amorphous calcium phosphate, and washing the amorphous calcium phosphate.

The method for preparing amorphous calcium phosphate and the oral care composition of the present invention may further comprise: a step (d2) after the step (d1): drying the amorphous calcium phosphate. Herein, the amorphous calcium phosphate may be dried by spray drying, or heating.

According to the method for preparing amorphous calcium phosphate and the oral care composition of the present invention, the sintering temperature in the step (b) may be 800-1200° C. Preferably, the sintering temperature in the step (b) is 1000-1100° C.

According to the method for preparing amorphous calcium phosphate and the oral care composition of the present invention, the sintering time in the step (b) may be 1-3 hrs.

According to the method for preparing amorphous calcium phosphate and the oral care composition of the present invention, the phosphate solution in step (d) may be disodium phosphate (Na₂HPO₄) solution or dipotassium phosphate (K₂HPO₄) solution.

Other objects, advantages, and novel features of the invention will become more apparent from the following detailed description when taken in conjunction with the accompanying drawings.

### BRIEF DESCRIPTION OF THE DRAWINGS

FIG. 1 is an X-ray diffraction diagram of the amorphous calcium phosphate prepared in accordance with Example 1 of the present invention;

FIG. 2 is an X-ray diffraction diagram of the amorphous calcium phosphate prepared in accordance with Example 1 and Comparative Examples 2-4 of the present invention;

FIG. 3 is a testing result showing the acid buffering capacities of water, and the amorphous calcium phosphate prepared in accordance with Example 1 and Comparative Example 1 of the present invention; and

FIG. 4 is a test result showing calcium ions release rate in water of the amorphous calcium phosphate prepared in accordance with Example 1 and Comparative Examples 1 and 5 of the present invention.

### DETAILED DESCRIPTION OF THE PREFERRED EMBODIMENT

The technical feature of the present invention will be described in more detail, accompanied with the following Examples.

### EXAMPLE 1

### Preparation of a Basic Calcium-Containing Solution

Oyster shells were cleaned completely, and the black parts were removed. After the oyster shells were pulverized, the powder was sintered under 1100° C. for 2 hours to obtain calcium-containing powders, and the main component of the calcium-containing powders was calcium oxide. Then 5 g of the calcium-containing powder was added into 1 L deionized water, followed by stirring, filtering, and collecting the basic filtrate to obtain a basic calcium-containing solution with high concentration.

### Preparation of ACP Powder

The 1M aqueous Na₂HPO₄ solution and aforementioned basic calcium-containing solution were mixed by stirring to precipitate a white product, i.e. amorphous calcium phosphate. Finally, the solid product was separated from liquid through filtration, followed by drying the solid product directly to obtain amorphous calcium phosphate powders.

### X-Ray Diffraction Data of the ACP Powder

The amorphous calcium phosphate powders prepared in the present example were analyzed by using an X-ray diffraction meter, and the test conditions are described as following:

Scan rate: 1°/min

Scan angle: 10-60°

Sample width: 0.05°/S

Div Slit: ½°

Div H.L.Slit: 5 mm

Sct Slit: auto

Rec Slit: 0.3 mm

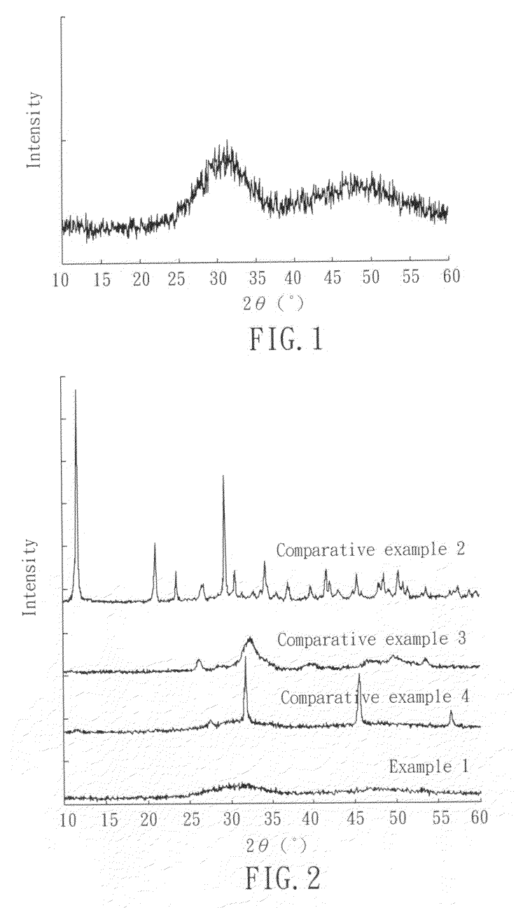

FIG. 1 shows the X-ray diffraction data, which indicates that the product prepared in the present example is amorphous calcium phosphate.

### Comparative Example 1

In the present comparative example, amorphous calcium phosphate was prepared by using the conventional process, which uses calcium chloride as a starting material. 1M Na₂HPO₄ solution and 1.67M calcium chloride solution were mixed by stirring to obtain amorphous calcium phosphate powders. During the process of mixing two solutions, a sodium hydroxide solution was used to adjust the pH of the reaction solution and control the pH between 8.5˜12. The reaction time was about 24 hours to stabilize the pH value of the reaction solution. Then, the solid product was separated from the reaction solution by filtration and washed using a suitable amount of deionized water to remove the NaCl generated from neutralization. Finally, the solid product was filtered again, followed by drying to obtain amorphous calcium phosphate powders.

### Comparative Example 2

### The pH of the Reaction Solution was Not Controlled

The method for preparing the amorphous calcium phosphate powder in Comparative Example 2 is the same as the method used in Comparative Example 1, except that the pH value of the reaction solution is 4.0.

### Comparative Example 3

### The pH of the Reaction Solution was Not Properly Controlled

The method for preparing the amorphous calcium phosphate powder in Comparative Example 3 is the same as the method used in Comparative Example 1, except that the pH of the reaction solution is 7.0.

### Comparative Example 4

### Salt Residue

The method for preparing the amorphous calcium phosphate powder in Comparative Example 4 is the same as the method used in Comparative Example 1, except that the solid product was not washed completely. Hence, some NaCl still remained in the amorphous calcium phosphate powders.

### Comparative Example 5

### The Starting Material was Calcium Oxide

5 g of CaO powder was added into 1 L deionized water, followed by stirring, filtering, and collecting the basic filtrate to obtain a basic calcium-containing solution with high concentration. Then, the method used for preparing calcium phosphate powder was performed. The method used for preparing calcium phosphate powder was the same as the method described in Example 1 to obtain amorphous calcium phosphate powders of the present comparative example.

### Testing Example

Comparison of the process of Example 1 with those of Comparative Examples 2-4

The powder products prepared in Example 1 and Comparative Examples 2-4 were analyzed with an X-ray diffraction meter. The test condition was the same as that described above, and the test result is shown in FIG. 2. After performing X-ray diffraction analysis, different forms of calcium phosphate were found in the powder product prepared by using Comparative Example 2. Hence, the product of Comparative Example 2 was not the amorphous calcium phosphate powders as expected. Furthermore, the X-ray diffraction analysis showed that the powder product prepared in Comparative Example 3 was crystalline calcium phosphate. The solubility of crystallized calcium phosphate is so low that diminishes the releasing rate of Ca²⁺ and PO₄³⁻. The crystalline calcium phosphate cannot be applied effectively to oral care compositions.

On the other hand, although the pH of the reaction solution was controlled properly in Comparative Example 4, the solid product was not washed appropriately, so salts still remained in the powder product. When the conventional method was used to prepare amorphous calcium phosphate, a suitable amount of water was needed to remove the salt generated from neutralization. If the amount of the washing water is too much, the form of calcium phosphate will be changed, as shown in Comparative Example 3. If the amount of the washing water is inadequate, metal salt will be detected in the product, as shown in Comparative Example 4. Hence, the water amount for cleaning will influence the quality of the products.

The X-ray diffraction data shows that the powder product prepared in Example 1 is amorphous calcium phosphate powder with high purity, and there are no contaminants in the amorphous calcium phosphate powders. When the method of Example 1 is used for preparing amorphous calcium phosphate, the problems of pH value and salt residues do not arise. Hence, amorphous calcium phosphate with high purity can be steadily generated in a simple and rapid way by use of the method described in Example 1.

### Element Analysis of Amorphous Calcium Phosphate Powders by Inductively Coupled Plasma Mass Spectroscopy (ICP-MS)

The amorphous calcium phosphate powders prepared in Example 1 and Comparative Example 5 were analyzed by ICP-MS, and the analysis results were shown in Table 1. The amorphous calcium phosphate powders prepared in Example 1 contains fluoride of 535.97 ppm and strontium of 168.10 ppm, but these two trace elements were not detected in the amorphous calcium phosphate powder prepared in Comparative Example 5.

TABLE 1

| | Sr (ppm) | F (ppm) |
|---|---|---|
| Example 1 | 168.10 | 534.97 |
| Comparative Example 5 | N.D. | N.D. |

(Note: N.D. means not detectable)

As shown in Table 1, the method of the present invention can synthesize amorphous calcium phosphate from oyster shells, and the trace elements contained in the oyster shells can be still kept in the product. In addition, trace elements of fluoride and strontium are not included in other amorphous calcium phosphate products prepared by general material and process.

### Test for the Buffering Capacity of Amorphous Calcium Phosphate

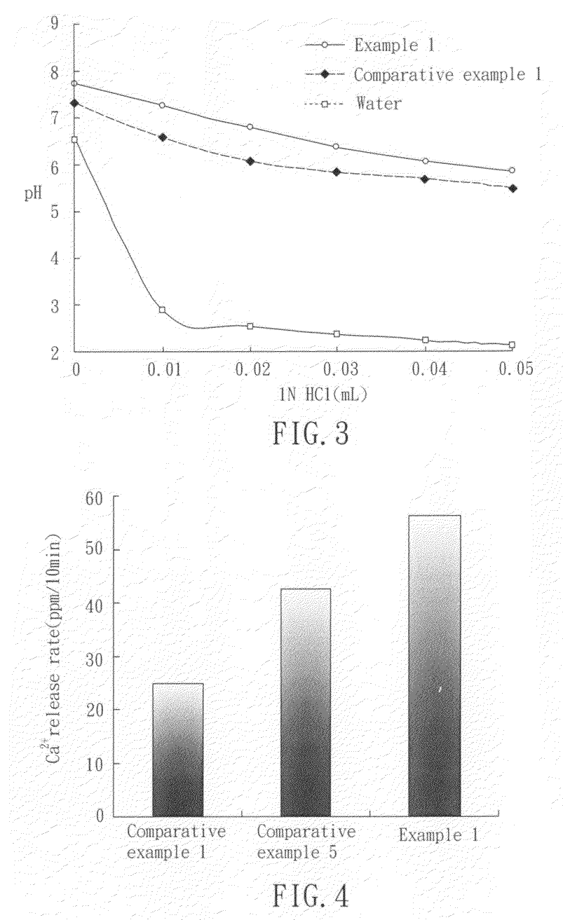

The powders prepared by using Example 1 and Comparative Example 1 were individually dissolved in deionized water to obtain 1 wt % amorphous calcium phosphate solution. At the same time, deionized water was used as a control group to compare with the aforementioned amorphous calcium phosphate solution. Then, 1 N hydrogen chloride solution was discharged into deionized water and amorphous calcium phosphate solution drop-by-drop, respectively. A pH meter was used to detect the pH change in each solution system. The pH changing cure represents the buffering capacity of each solution and the test results are summarized in FIG. 3. The slope of the pH changing rate represents the buffer slope of each solution and the results are listed in Table 2.

TABLE 2

| | | Comparative | |
|---|---|---|---|
| Intercept = 7 | Example 1 | Example 1 | Water |
| Buffering slope | −26 | −43 | −411 |
| (ΔpH/0.01 mL 1N HCl) | | | |

When hydrogen chloride solution was gradually dropped into deionized water, the pH of deionized water decreased from 6.5 to 2.1, and the buffering slope thereof was −411 (ΔpH/0.01 mL 1N HCl). When hydrogen chloride solution was dropped into the amorphous calcium phosphate solution formulated by the amorphous calcium phosphate powder prepared in Comparative Example 1, the pH of the amorphous calcium phosphate solution decreased from 7.4 to 5.5, and the buffer slope thereof was −43 (ΔpH/0.01 mL 1N HCl). However, when hydrogen chloride solution was titrated into the amorphous calcium phosphate solution formulated by the amorphous calcium phosphate powder prepared in Example 1, the pH value of the amorphous calcium phosphate solution decreased from 7.7 to 5.9, and the buffer slope thereof was only −26 (ΔpH/0.01 mL 1N HCl). According to the aforementioned results, the amorphous calcium phosphate powder prepared by using Example 1 exhibits excellent buffering effect compared with deionized water as well as amorphous calcium phosphate powder prepared in Comparative Example 1.

It is feasible to reduce the acidity in mouth fluid and decrease the incidence rate of caries when the amorphous calcium phosphate powders prepared by using Example 1 are applied. The oral care composition containing amorphous calcium phosphate powders from Example 1 shows effective buffering effect on dental care. In addition, the amorphous calcium phosphate powder from the method of Example 1 contains strontium (Sr) and fluoride (F) which is different from the amorphous calcium phosphate powder prepared by the conventional method.

### Calcium Ion Release Rate of Amorphous Calcium Phosphate

The amorphous calcium phosphate powders prepared from Example 1, Comparative Example 1, and Comparative Example 5 were dissolved under a ratio of 2 mg powder/10 mL deionized water into a solution, respectively. After 10 min, calcium ion electrode was used to detect concentration change to represent calcium ion releasing rate. The test result is shown in FIG. 4. The Ca²⁺ concentration was 56 ppm in the amorphous calcium phosphate solution of Example 1, only 24 ppm in the amorphous calcium phosphate solution of Comparative Example 1, and 42 ppm in the amorphous calcium phosphate solution of Comparative Example 5. Comparing the effect of Example 1 with that of Comparative Example 1, the Ca²⁺ concentration increased 127%. Also, comparing the effect of Example 1 with that of Comparative Example 5, the Ca²⁺ concentration increased 32%. These results indicate that the amorphous calcium phosphate powders prepared by using Example 1 has excellent Ca²⁺ release rate, so it is possible to supply Ca²⁺ in mouth to enhance the remineralization on teeth.

In conclusion, the method for preparing amorphous calcium phosphate and the oral care composition containing amorphous calcium phosphate prepared by the same of the present invention has the following advantages. (1) The method of the present invention can fully utilize the abundant calcium content in the oyster shells and reduce the cost of manufacturing amorphous calcium phosphate. The oyster shells can be reused and reduced environment pollution. (2) The method of the present invention is very simple, and the time for preparing amorphous calcium phosphate can be shorted down to about 5 min. However, the reaction time for conventional method is about 24 hours. (3) The yield of amorphous calcium phosphate is about 90% by the method of the present invention. (4) The amorphous calcium phosphate powder grain prepared by the method of the present invention is smaller and more transparent than that prepared by using the conventional method. Hence, the dissolution rate of the amorphous calcium phosphate powder can be increased. (5) The method of the present invention does not need alkaline solution to maintain the pH of the reaction solution, so the problem of the presence of salt residue can be eliminated. (6) The oral care composition of the present invention contains trace elements, such as strontium (Sr) and fluoride (F), which can improve the effectiveness of dental care. (7) The amorphous calcium phosphate powder prepared by the method of the present invention has good buffering capacity. Hence, it is possible to neutralize the acidity in mouth fluid and prevent caries. (8) The amorphous calcium phosphate powder prepared by the method of the present invention has excellent Ca²⁺ release rate, so it is possible to enhance the remineralization on teeth.

### INDUSTRIAL APPLICATIONS

According to the method of the present invention, the starting material is oyster shell, so the problem of environment pollution caused by wasted oyster shells can be resolved and the manufacturing process is relatively simple. In addition, the method of the present invention does not need any alkaline solution during the process of synthesizing amorphous calcium phosphate, and the starting materials (sintered oyster shells and phosphate) are very cheap. Hence, the cost of producing amorphous calcium phosphate can be reduced greatly. Furthermore, the method of the present invention does not comprise steps of washing products; therefore, the wastewater discharge can be reduced, whereby environment pollution is accordingly reduced. Hence, when the amorphous calcium phosphate prepared by the method of the present invention is applied to the oral care composition, such as toothpaste and mouthwash, not only the production cost can be reduced, but also the effects of caries prevention and remineralization on teeth can be achieved. In addition, the amorphous calcium phosphate prepared by the present invention can further be added into food products, such as chewing gum, calcium-containing drinks, and calcium-containing food, in order to supply calcium and neutralize the acidity in mouth.

Although the present invention has been explained in relation to its preferred example, it is to be understood that many other possible modifications and variations can be made without departing from the scope of the invention as hereinafter claimed.

What is claimed is:

1. A method for preparing amorphous calcium phosphate, comprising the following steps:
(a) cleaning and pulverizing oyster shells;
(b) sintering the oyster shell to obtain a calcium-containing powder;
(c) dissolving the calcium-containing powder in water to obtain a calcium-containing solution; and
(d) mixing the calcium-containing solution with a phosphate-containing solution, to produce amorphous calcium phosphate.

2. The method as claimed in claim 1, further comprising a step (c1) after the step (c): filtering the calcium-containing solution.

3. The method as claimed in claim 1, further comprising a step (d1) after the step (d): filtering the amorphous calcium phosphate, and washing the amorphous calcium phosphate.

4. The method as claimed in claim 3, further comprising a step (d2) after the step (d1): drying the amorphous calcium phosphate.

5. The method as claimed in claim 4, wherein the amorphous calcium phosphate is dried by spray drying, or heating in the step (d2).

6. The method as claimed in claim 1, wherein the sintering temperature in the step (b) is 800-1200° C.

7. The method as claimed in claim 1, wherein the sintering temperature in the step (b) is 1000-1100° C.

8. The method as claimed in claim 1, wherein the sintering time in the step (b) is 1-3 hrs.

9. The method as claimed in claim 1, wherein the phosphate solution in step (d) is Na₂HPO₄ solution or K₂HPO₄ solution.

10. An oral care composition, comprising:
amorphous calcium phosphate;
wherein the amorphous calcium phosphate is prepared in accordance with the following steps:
(a) cleaning and pulverizing oyster shells;
(b) sintering the oyster shell to obtain a calcium-containing powder;
(c) dissolving the calcium-containing powder in water to obtain a calcium-containing solution; and
(d) mixing the calcium-containing solution with a phosphate-containing solution, to produce amorphous calcium phosphate containing strontium (Sr) and fluoride (F).

11. The oral care composition as claimed in claim 10, further comprising a step (c1) after the step (c): filtering the calcium-containing solution.

12. The oral care composition as claimed in claim 10, further comprising a step (d1) after the step (d): filtering the amorphous calcium phosphate, and washing the amorphous calcium phosphate.

13. The oral care composition as claimed in claim 12, further comprising a step (d2) after the step (d1): drying the amorphous calcium phosphate.

14. The oral care composition as claimed in claim 13, wherein the amorphous calcium phosphate is dried by spray drying, or heating in the step (d2).

15. The oral care composition as claimed in claim 10, wherein the sintering temperature in the step (b) is 800-1200° C.

16. The oral care composition as claimed in claim 10, wherein the sintering temperature in the step (b) is 1000-1100° C.

17. The oral care composition as claimed in claim 10, wherein the sintering time in the step (b) is 1-3 hrs.

18. The oral care composition as claimed in claim 10, wherein the phosphate solution in step (d) is Na₂HPO₄ solution or K₂HPO₄ solution.
